(19) United States
(12) Patent Application Publication
(10) Pub. No.: US 2024/0307111 A1
(43) Pub. Date: Sep. 19, 2024

(54) DEVICES AND METHODS FOR DETERMINING RENAL ARTERIOLAR VASOMOTION

(71) Applicant: MEDTRONIC IRELAND MANUFACTURING UNLIMITED COMPANY, Dublin 2 (IE)

(72) Inventors: Douglas A. Hettrick, Andover, MN (US); Paul J. Coates, Corte Madera, CA (US)

(21) Appl. No.: 18/264,995

(22) PCT Filed: Feb. 22, 2022
(86) PCT No.: PCT/IB2022/051545

§ 371 Date: Aug. 10, 2023

Related U.S. Application Data
(60) Provisional application No. 63/153,162, filed on Feb. 24, 2021.

(51) Int. Cl. A61B 18/14; A61B 18/00; A61B 18/02; A61B 18/12

(52) U.S. Cl. CPC A61B 18/1492; A61B 18/02; A61B 18/1206; A61B 2018/00214; A61B 2018/00434; A61B 2018/00577; A61B 2018/00821; A61B 2018/00875; A61B 2018/0212; A61B 2018/1435; A61B 2018/1467

(57) ABSTRACT

Methods, systems, devices, assemblies and apparatuses for renal denervation. The therapeutic assembly includes a sensor configured to detect a temperature or an impedance at a location within a vessel over a period of time. The therapeutic assembly includes a processor coupled to the sensor. The processor is configured to correlate the temperature or the impedance to a flow of blood within the vessel or an arterial pressure of the blood within the vessel. The processor is configured to determine vasomotion of a wall of a distal vessel based on the flow of blood or the arterial pressure of the blood.

This application claims priority to U.S. Provisional Application Ser. No. 63/153,162, filed Feb. 24, 2021, the entire content of which is incorporated by reference.

### TECHNICAL FIELD

This specification relates to a system, a device, a method and/or an apparatus for determining or calculating renal arteriolar vasomotion.

### BACKGROUND

Renal denervation is a minimally invasive procedure to treat resistant hypertension. During renal denervation, a nurse, doctor, technician or other hospital staff (or “clinician”) uses stimuli or energy, such as radiofrequency, ultrasound, cooling or other energy, to perform ablation within the renal arteries. This reduces activity of the nerves surrounding the vessel, which has been shown to result in a decrease in blood pressure and other benefits. The clinician uses the renal denervation device to deliver the stimuli or energy to the treatment site, e.g., through one or more energy delivery elements of the renal denervation device.

The renal denervation device may deliver neuromodulation energy, such as radiofrequency (RF) energy, through the one or more energy delivery elements to the treatment site, which heats the wall of the vessel, and as a consequence, warms the energy delivery element in contact with the wall of the vessel. There is currently no practical process to establish when the renal denervation (RDN) procedure is complete, and thus, a clinician relies on the empirical “manufacturer recommendations” to determine when to discontinue the RDN procedure regardless of the type of the energy source. Moreover, some processes have been proposed to detect the completion of denervation during the RDN procedure, but none has been proven to be simultaneously safe and practical.

Accordingly, there is a need for a system, apparatus, device and/or method to detect the successful completion of renal denervation safely and reliably.

### SUMMARY

In general, one aspect of the subject matter described in this specification is embodied in a therapeutic assembly for renal denervation. The therapeutic assembly includes a sensor configured to detect a temperature or an impedance at a location within a vessel over a period of time. The therapeutic assembly includes a processor coupled to the sensor. The processor is configured to correlate the temperature or the impedance to a flow of blood within the vessel or an arterial pressure of the blood within the vessel. The processor is configured to determine vasomotion of a wall of a distal vessel based on the morphometry of the flow of blood and/or the arterial pressure of the blood.

These and other embodiments may optionally include one or more of the following features. The processor may be configured to determine a frequency component of the temperature or the impedance at the location of the wall of the vessel over the period of time or the power spectral components of the combined signals. The processor may be configured to correlate the temperature or the impedance to the flow of blood within the vessel or the arterial pressure of the blood within the vessel further based on the frequency of the temperature or the impedance.

The therapeutic assembly may include an energy delivery element. The energy delivery element may be configured to deliver neuromodulation energy to the location of the wall of the vessel. The processor may be configured to compare the vasomotion of the wall prior to the delivery of the neuromodulation energy and the vasomotion of the wall after the delivery of the neuromodulation energy. The processor may be configured to determine a success or failure of renal neuromodulation based on the comparison.

The processor may be configured to determine the vasomotion of the wall of the vessel based on the flow of blood and/or the arterial pressure of the blood prior to the delivery of the neuromodulation energy. The processor may be configured to correlate the temperature to the flow of blood. The processor may be configured to measure or detect, using the sensor, variations in the temperature at the location of the wall within the vessel over the period of time. The processor may be configured to associate the variations in the temperature with the flow of blood. The variations in the temperature may be inversely proportional to the rate of flow of blood.

The processor may be configured to correlate the impedance to the arterial pressure of the blood within the vessel. The processor may be configured to measure or detect, using a sensor, variations in the impedance at the location of the wall within the vessel over the period of time. The processor may be configured to associate the variations in the impedance with the arterial pressure of the blood within the vessel. The variations in the impedance may be inversely proportional to a diameter of the vessel that corresponds to the arterial pressure of the blood.

In another aspect, the subject matter is embodied in a therapeutic assembly for renal denervation. The therapeutic assembly includes a first sensor configured to detect a temperature at a location of a wall of a vessel over a period of time. The therapeutic assembly includes a second sensor configured to detect an impedance at the location of the wall of the vessel over the period of time. The therapeutic assembly includes a processor coupled to the first sensor and the second sensor. The processor is configured to correlate the temperature to a flow of blood within the vessel. The processor is configured to correlate the impedance to an arterial pressure of the blood within the vessel. The processor is configured to determine vasomotion of the wall of the vessel based on the flow of blood and the arterial pressure of the blood.

In another aspect, the subject matter is embodied in a method of renal denervation. The method includes detecting, by a first sensor, a temperature at a location of a wall of a vessel over a period of time. The method includes detecting, by a second sensor, an impedance at the location of the wall of the vessel over the period of time. The method includes correlating, by a processor, the temperature to a flow of blood within the vessel. The method includes correlating, by the processor, the impedance to an arterial pressure of the blood within the vessel. The method includes determining, by the processor, vasomotion of the wall of the vessel based on the flow of blood and the arterial pressure of the blood.

### BRIEF DESCRIPTION OF THE DRAWINGS

Other systems, methods, features, and advantages of the present invention will be or will become apparent to one of ordinary skill in the art upon examination of the following figures and detailed description. It is intended that all such additional systems, methods, features, and advantages be included within this description, be within the scope of the present invention, and be protected by the accompanying claims. Component parts shown in the drawings are not necessarily to scale and may be exaggerated to better illustrate the important features of the present invention. In the drawings, like reference numerals designate like parts throughout the different views.

FIG. 1 shows an example conceptual illustration of the therapeutic assembly according to an aspect of the invention.

FIG. 2A shows an example renal denervation device of the therapeutic assembly of FIG. 1 in a low-profile delivery configuration according to an aspect of the invention.

FIG. 2B shows an example renal denervation device of the therapeutic assembly of FIG. 1 in an expanded deployed configuration according to an aspect of the invention.

FIG. 3 shows an example renal denervation device of the therapeutic assembly of FIG. 1 in the expanded deployed configuration within a blood vessel according to an aspect of the invention.

FIG. 4 shows a front view of the distal portion of the renal denervation device of the therapeutic assembly of FIG. 1 that looks down a longitudinal axis of the blood vessel according to an aspect of the invention.

FIG. 5 is a block diagram of an example generator of the therapeutic assembly of FIG. 1 according to an aspect of the invention.

FIG. 6 is a flow diagram of a process for determining or calculating renal sympathetic nerve vasomotion using the therapeutic assembly of FIG. 1 according to an aspect of the invention.

FIG. 7 is a flow diagram of a process FIG. 7 is a flow diagram of a process for determining whether renal neuromodulation is complete and/or was successful using the therapeutic assembly of FIG. 1 according to an aspect of the invention.

FIG. 8 shows a graph of an example measured temperature and impedance at a treatment site using the therapeutic assembly of FIG. 1 according to an aspect of the invention.

FIG. 9A shows a graph of an example measured blood flow after applying neuromodulation energy using the therapeutic assembly of FIG. 1 according to an aspect of the invention.

FIG. 9B shows a graph of an example measured arterial pressure after applying neuromodulation energy using the therapeutic assembly of FIG. 1 according to an aspect of the invention.

### DETAILED DESCRIPTION

Disclosed herein are systems, devices, methods and/or apparatuses for a therapeutic assembly including a renal denervation device that delivers neuromodulation energy from one or more energy delivery elements and measures, detects or otherwise obtains parameters related to renal arteriolar vasomotion. The renal sympathetic nerves are an underappreciated controller of renal blood flow, and as such, renal sympathetic vasomotion may be a biomarker for therapeutic renal denervation. The quantification of the renal sympathetic vasomotion, which measures the rhythmic constriction and relaxation of blood vessels, allows for more accurate treatment of hypertension. For example, the therapeutic assembly may measure, detect or otherwise obtain the temperature of the energy delivery element or treatment site and/or the impedance between two or more energy delivery elements or an energy delivery element and another sensor to more accurately determine in real-time when to discontinue or terminate ablation and/or whether the RDN procedure is targeting the proper treatment site within the vessel and/or delivering the proper amount of energy.

Other benefits and advantages of the therapeutic assembly include having built-in sensors that measure, detect or otherwise obtain parameters related to renal arteriolar vasomotion. Since the sensors are built-in or included within the therapeutic assembly a separate additional device to detect, measure or obtain the parameters is unnecessary, which reduces complexity and costs of the RDN procedure. Additionally, since the clinician does not need to monitor a separate device, and then communicate to terminate or discontinue treatment, the termination of treatment is automatic and/or nearly immediately performed, which reduces delay in reacting to when the treatment should be discontinued. This prevents overtreatment to the treatment location.

Moreover, since the sensors are built-in or included within the therapeutic assembly the detection may occur in real-time simultaneously or concurrently with the renal neuromodulation and/or may occur immediately after the renal neuromodulation so that the affects of the renal neuromodulation are measured, detected or obtained without delay, and thus, result in a more accurate and precise measurement of the affect of the renal neuromodulation.

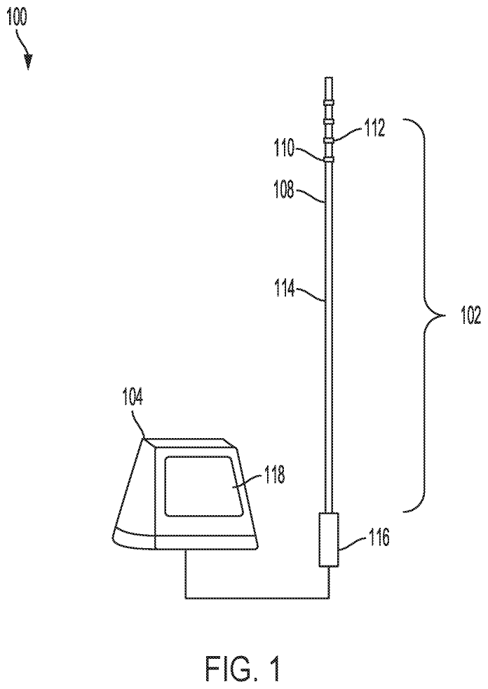

FIG. 1 shows the therapeutic assembly 100. The therapeutic assembly 100 performs renal denervation within the renal artery of a human patient. Renal denervation is a minimally invasive procedure to treat resistant hypertension. The therapeutic assembly 100 includes a renal denervation device 102 and/or a generator 104. The renal denervation device 102 may include any device that delivers energy or stimulus to a target nerve within a wall of a blood vessel, such as the renal nerve of the renal artery. The energy or stimulus may include, for example, at least one of a radio frequency stimulus, a thermal stimulus, a cryogenic stimulus, a microwave stimulus, an ultrasonic stimulus or other form of energy or stimulus.

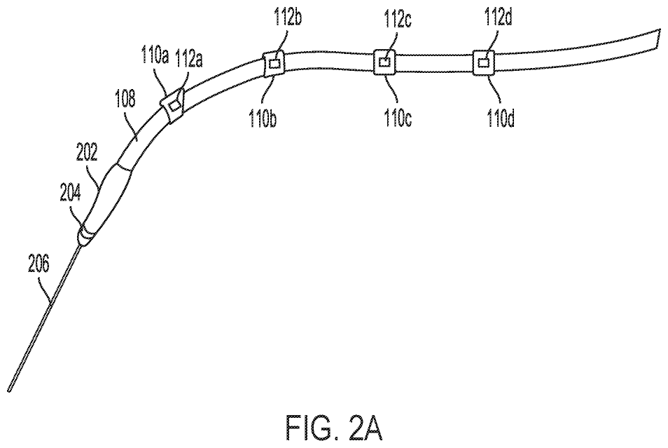

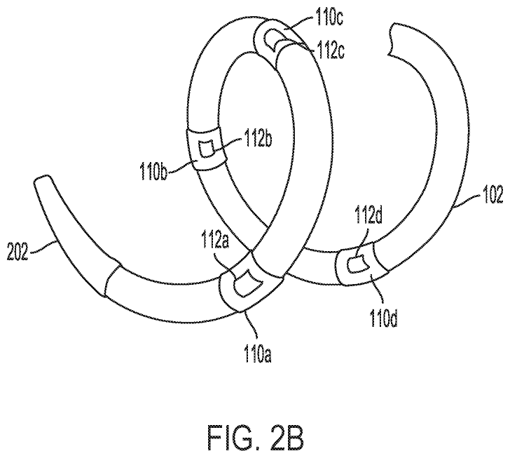

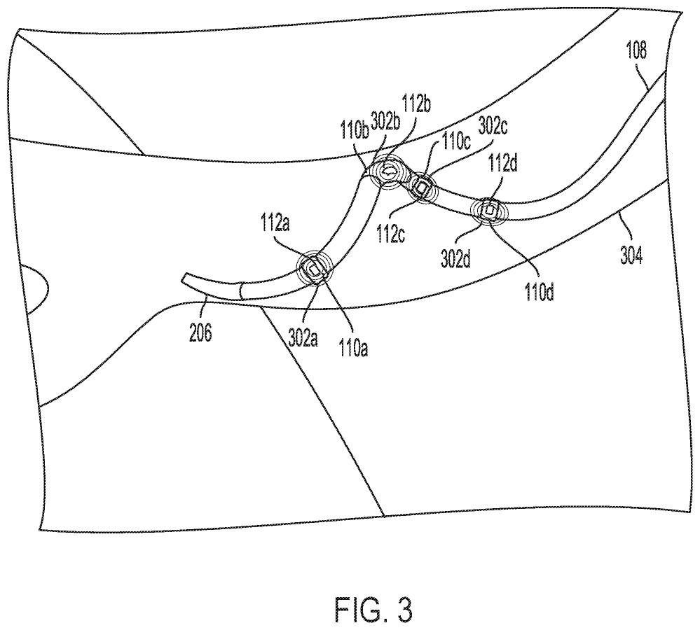

The renal denervation device 102 has a catheter 108, one or more energy delivery elements 110, such as an electrode, and/or one or more sensors 112. The renal denervation device 102 may have an elongated shaft 114 with a handle 116. The elongated shaft 114 with the handle 116 may be used to guide and/or advance a distal portion of the catheter 108 through the blood vessels of the patient, such as a human patient, to a target location of a blood vessel and remotely manipulate the distal portion of the catheter 108. The catheter 108 may be intravascularly delivered into the patient, e.g., a blood vessel of the patient, in a low-profile configuration, such as the substantially straight configuration shown in FIG. 1. The catheter 108 may be over a meter in length. Upon delivery to a target location within and along the blood vessel, the catheter 108 may be deployed into an expanded deployed configuration, such as a generally helical or spiral configuration or other suitable configuration, which the one or more energy delivery elements 110, such as one or more electrodes, may contact the blood vessel, as shown in FIG. 3 for example. In the expanded deployed state, the renal denervation device 102 may deliver energy at a treatment site and provide therapeutically-effective electrically and/or thermally induced denervation to a nerve within the wall of the blood vessel. The catheter 108 may be deployed in a generally helical or spiral configuration that rotates clockwise or counter-clockwise relative to a longitudinal central axis. FIGS. 2A-2B show the deployment of the renal denervation device 102. In particular, FIG. 2A shows the catheter 108 in the low-profile configuration, and FIG. 2B shows the catheter 108 in the expanded deployed configuration.

The catheter 108 may have a distal tip 202. The distal tip 202 points into the lumen of the blood vessel. The distal tip 202 may have a high density marker band 204. The high density marker band 204 allows a clinician to identify the distal tip 202 of the catheter 108 under fluoroscopy. The distal portion of the catheter 108 may be approximately 4 cm-5 cm in length, and the distal tip 202 may be approximately 1 cm-2 cm in length.

The catheter 108 may have a wire 206 within the lumen of the catheter 108. The distal tip 202 allows the wire 206 to extend out and away from the distal tip 202 when the catheter 108 is in the low-profile configuration and to be advanced through the blood vessels to the target location of the blood vessel. When the wire 206 is retracted within the distal tip 202 and into the catheter 108, the catheter 108 changes shape from the low-profile configuration, such as the substantially straight configuration, as shown in FIG. 2A for example, to the expanded deployed configuration, such as a generally helical or spiral configuration, as shown in FIG. 2B for example.

The renal denervation device 102 has one or more energy delivery elements 110. The one or more energy delivery elements 110 may include an electrode, such as a radiofrequency (RF) electrode, a radiofrequency (RF) probe, a thermal probe, a cryogenic probe, a microwave probe, an ultrasonic probe, an optical source or a chemical injector. The one or more energy delivery elements 110 may be positioned on the distal portion of the catheter 108. The one or more energy delivery elements 110 may include multiple energy delivery elements 110, such as the energy delivery elements 110a-d, as shown in FIGS. 2A, 2B and 3 for example, or any other N number of energy delivery elements 110. The energy delivery elements 110a-d may be arranged approximately 90 degrees apart relative to a longitudinal axis that runs through the center of the catheter 108 when in the spiral configuration. The energy delivery elements 110 may be spaced any suitable distance from each other, and the spacing may vary based on the application of the therapeutic assembly 100 and its intended use.

When there are multiple energy delivery elements 110, each energy delivery element 110 may deliver power independently, either simultaneously, selectively, and/or sequentially, to a treatment site. The multiple energy delivery elements 110 may deliver power among any desired combination of the one or more energy delivery elements 110.

The one or more energy delivery elements 110 may be introduced into and advanced along a blood vessel, such as the renal artery and may be positioned to contact the blood vessel in the expanded deployed configuration at different intervals and/or locations along the wall of the blood vessel. For example, a first energy delivery element 110a may contact the wall of the blood vessel 304 at a first location 302a, a second energy delivery element 110b may contact the wall of the blood vessel 304 at a second location 302b, a third energy delivery element 110c may contact the wall of the blood vessel 304 at a third location 302c and a fourth energy delivery element 110d may contact the wall of the blood vessel 304 at a fourth location 302d. The renal denervation device 102 may deliver energy through the one or more energy delivery elements 110 at the treatment sites and provide therapeutically-effective electrically- and/or thermally-induced denervation.

The renal denervation device 102 may include one or more sensors 112. The one or more sensors 112 may measure one or more parameters at or near the treatment site. The one or more parameters may include a temperature of one or more energy delivery elements 110 and/or a temperature at or near the treatment site. For example, the one or more sensors 112 may be a temperature sensor that measures the temperature at a location of the wall of the blood vessel. In another example, the one or more sensors 112 may be another type of sensor that measures a different parameter, such as an impedance, pressure, optical, flow, or amount of chemical.

Each of the one or more sensors 112 may be coupled or integrated with a corresponding one of the one or more energy delivery elements 110. For example, the sensor 112a may be integrated with energy delivery element 110a, the sensor 112b may be integrated with energy delivery element 110b, the sensor 112c may be integrated with energy delivery element 110c and the sensor 112d may be integrated with energy delivery element 110d, as shown in FIG. 2B for example.

The one or more sensors 112 may be proximate to or within a corresponding energy delivery element 110. The one or more sensors 112 may be sufficiently close or proximate to the corresponding energy delivery element 110 so that the one or more sensors 112 experience an increase in tissue temperature induced by the energy delivery element 110. The increased tissue temperature may be an amount that is approximately equal to or is lower than a therapeutically-effective amount so long as there is a measurable temperature increase in proximity to the one or more sensors 112. The delivery of the energy may be a continuous delivery of an amount below a therapeutic-effective amount or pulses of higher amounts of energy.

In some implementations, the one or more sensors 112 may be integrated with the one or more energy delivery elements 110. For example, the energy delivery element 110 may be an electrode, which has two wires. One wire may be made from copper and the other may be made from a copper-nickel alloy. The wires may both transmit the signal from the sensor 112 and also convey the energy to the energy delivery element 110.

The signal may be a temperature signal that indicates the temperature of the blood vessel. The two wires may measure the temperature through a thermocouple effect. The two wires may have a voltage gap, and as the temperature changes due to the change in blood flow at the treatment site, the amount of voltage across the voltage gap changes. For example, when there is more blood flowing at the treatment site, there is a cooling effect, which results in a decrease in temperature, and when there is less blood flowing at the treatment site, there is a warming effect, which results in an increase in temperature. The cooling and/or the warming effect may only occur if energy is being delivered to the treatment site and/or to the tissue at or near the treatment site so that the tissue temperature is different than the body temperature. For example, the tissue temperature may be elevated when the thermal energy or RF energy is being delivered to the treatment site and/or to the tissue at or near the treatment site and may be depressed when cryo-therapeutic energy is being delivered. The amount of voltage across the voltage gap may be measured and may be associated to a temperature at the treatment site.

In some implementations, the one or more sensors 112 may measure the temperature at an independent location away from the one or more energy delivery elements 110. For example, the therapeutic assembly 100 may have an independent heating element or other additional device in place solely to be used to measure the temperature variation associated with the changes in the blood flow. This heating element may be independent of the one or more energy delivery elements 110 that heat the tissue, and as such, the renal denervation device 102 may determine the renal sympathetic vasomotion of the blood vessel even when the neuromodulation energy is not being delivered to the tissue, e.g., a continuous measurement of the temperature, which corresponds to the renal sympathetic vasomotion. This additional device may have an appendage that branches off or may have the sensor be attached to the outside of the catheter 108 and may be connected back and integrated with the power source.

The one or more sensors 112 may measure or calculate an impedance at or near the treatment site. The impedance may be measured from one energy delivery element 110 to another energy delivery element 110 and/or from one energy delivery element 110 to another sensor, such as a grounding patch.

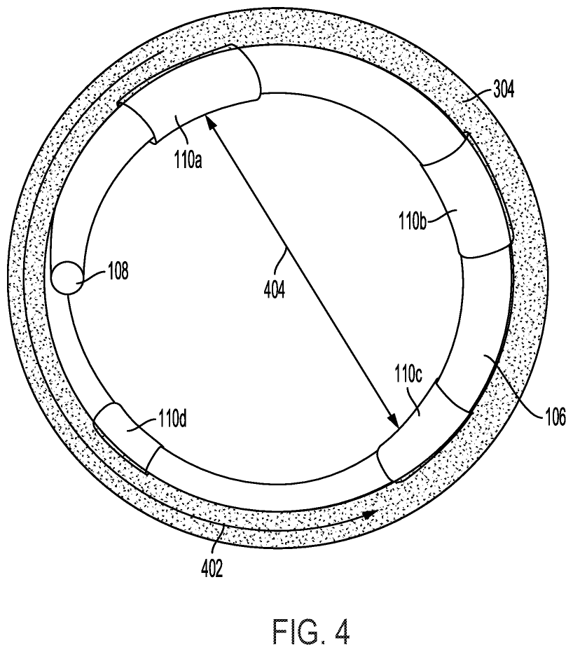

FIG. 4 shows a front view of the distal portion of the renal denervation device 102 that looks down a longitudinal axis of the blood vessel 304. For example, one of the energy delivery elements 110a-d may generate a signal and propagate the signal between the one or more energy delivery elements 110a-d through a path in the wall of the blood vessel 304, as indicated by the arrow 402, or through the blood or other medium in the lumen of the blood vessel 304, as indicated by the arrow 404. Moreover, the same signal can be detected at multiple ones of the energy delivery elements 110, at the same energy delivery element 110 that generates the signal and/or at another sensor, such as a grounding patch. The degree of attenuation of the signal, the difference in the propagation time of the signal and/or other parameters of the signal may be correlated to the amount of blood between the energy delivery elements 110, the distance, the rate and/or the conduction path of the signal that travelled through the blood vessel, which may relate to the dimension of the blood vessel 304. Changes in those parameters may also correlate and/or correspond to changes in the dimension of the blood vessel 304. Changes in the vessel diameter may be directly correlated or proportionate to changes in blood pressure, since the artery is compliant.

In some implementations, the therapeutic assembly 100 may use the “four electrode” technique where the proximal and distal energy delivery elements 110 drive a constant current field and the voltage is measured between the two electrodes to determine the impedance of the vessel segment bounded by the two energy elements 110. For example, the one or more sensors 112 may measure the impedance from a first energy delivery element to a second energy delivery element, such as from an ablation electrode to a dispersive electrode. In some implementations, the therapeutic assembly 100 may measure the voltage across the voltage gap of the two wires and calculate the impedance from the current and measured voltage.

The therapeutic assembly includes a generator 104. The generator 104 may be a radio frequency generator or other generator that delivers a denervation stimulus or energy through the one or more energy delivery elements 110 to the wall of the blood vessel at the treatment location. The denervation stimulus may include a non-electric stimulus, for example, a chemical agent, optical stimulus, a thermal stimulus, a cooling stimulus, a microwave stimulus or other form of stimuli. The generator 104 may have a cable, an electrical lead and/or wire that is electrically conductive and runs through the catheter 108 within a lumen and is electrically coupled with the one or more energy delivery elements 110. In some implementations, the generator 104 may have separate leads and/or wires that electrically couple with a corresponding energy delivery element 110 of the one or more energy delivery elements 110 so that each energy delivery element 110 may operate independently of the others. For example, the generator 104 may have multiple separate channels, such as four radio frequency (RF) channels to deliver RF energy independently to the one or more energy delivery elements 110a-d and control and monitor each energy delivery element 110a-d independently. The generator 104 may generate energy that ultimately is transmitted through the electrical lead to the one or more energy delivery elements 110.

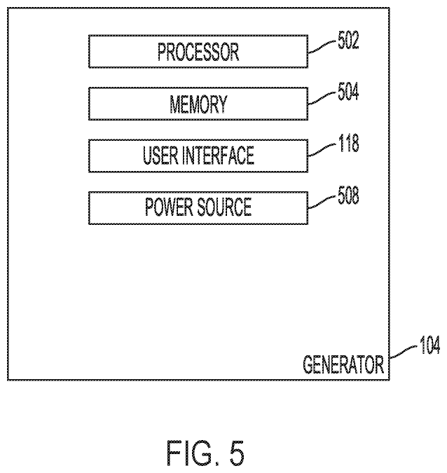

The generator 104 may have one or more processors 502, a memory 504, a user interface 118 and/or a power source 508, as shown in FIG. 5 for example. The one or more processors 502 may be electrically coupled to the memory 504, the user interface 118 and/or the power source 508. The one or more processors 502 may include one or more controllers that obtain a temperature signal and/or an impedance signal that indicates the temperature and/or impedance at the treatment site, respectively, and determines the parameters related to the renal sympathetic vasomotion of the blood vessel based on the temperature signal and/or impedance signal. Once the renal sympathetic vasomotion is determined, the one or more processors 502 may control a state of each of the one or more energy delivery elements 110 and the amount of energy delivered to each of the one or more energy delivery elements 110 by the power source 508 and/or may provide an indication to the clinician. The one or more processors 502 may be coupled to the memory 504 and execute instructions that are stored in the memory 504.

The generator 104 may have a memory 504. The memory 504 may be coupled to the one or more processors 502 and store instructions that the one or more processors 502 executes. The memory 504 may include one or more of a Random Access Memory (RAM), Read Only Memory (ROM) or other volatile or non-volatile memory. The memory 504 may be a non-transitory memory or a data storage device, such as a hard disk drive, a solid-state disk drive, a hybrid disk drive, or other appropriate data storage, and may further store machine-readable instructions, which may be loaded and executed by the one or more processors 502.

The generator 104 may have a power source 508, such as a RF generator or other electrical source. The power source 508 provides a selected form and magnitude of energy for delivery to the treatment site via the renal denervation device 102. The generator 104 may have a user interface 118. The generator 104 may receive input, such as the selected form and the magnitude of energy to be delivered to each of the one or more energy delivery elements 110 and/or an indication to terminate or discontinue energy delivery, via the user interface 118.

The user interface 118 may include an input/output device that receives user input from a user interface element, a button, a dial, a microphone, a keyboard, or a touch screen. The user interface 118 may provide an output to an output device, such as a display, a speaker, an audio and/or visual indicator, or a refreshable braille display. The output device may display an alert or notification or other information to the clinician and/or to confirm status and/or commands from the clinician. The output device may be an audio output device that outputs an audio indicator that indicates the notification or information to be provided to the clinician.

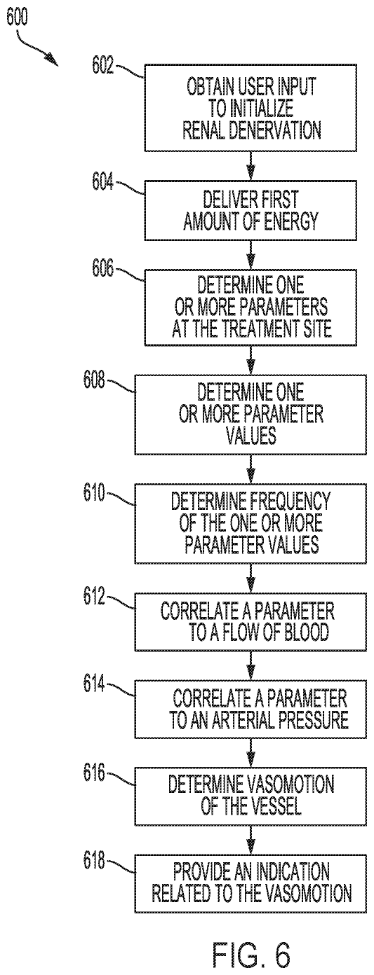

FIG. 6 is a flow diagram of a process 600 for determining or calculating renal sympathetic nerve vasomotion. One or more computers or one or more data processing apparatuses, for example, the processor 502 of generator 104 of the therapeutic assembly 100 of FIG. 1, appropriately programmed and/or using one or more other components, such as the one or more sensors 112 and/or the one or more energy delivery elements 110, may implement the process 600.

The therapeutic assembly 100 may include a generator 104, which controls the delivery of energy to the one or more energy delivery elements 110 of the renal denervation device 102. The generator 104 of the therapeutic assembly 100 receives user input that indicates initialization of the renal denervation device 102 (602). The generator 104 may receive the user input, which may be an indication to power-on the generator 104, via the user interface 118, which causes the generator 104 to power-on or initialize to deliver energy to the renal denervation device 102 and through the one or more energy delivery elements 110 to the wall of the blood vessel 304.

Once powered-on, the therapeutic assembly 100 may deliver a first amount of energy through the one or more energy delivery elements 110 (604). The first amount of energy may be reflective of a stand-by mode that delivers a low amount of energy to the treatment site to generate temperature fluctuations at the treatment site and/or at the energy delivery element 110. The low amount of energy may provide a low amount of heat at the treatment site that results in the temperature fluctuations at the treatment site and/or at the energy delivery element 110, which are measurable by the one or more sensors 112 that are proximate to the treatment site and/or at the energy delivery element 110.

The generator 104 may linearly or non-linearly ramp up or increase the amount of energy to the first amount of energy during the start-up phase until the amount of energy reaches the first amount of energy. The generator 104 may deliver the first amount of energy to each of the multiple energy delivery elements 110a-b when there are multiple energy delivery elements 110a-b. The multiple energy delivery elements 110a-d may be arranged to contact the wall of the blood vessel at approximately 90 degree angles relative to a longitudinal axis that runs through the center of the spiral or helical configuration when there are four energy delivery elements 110a-d and the catheter 108 is in the expanded deployed state within the blood vessel, as shown in FIG. 3 for example. The arrangement of the multiple energy delivery elements 110 may depend on the number of energy delivery elements 110. For example, when there are only three energy delivery elements 110, the energy delivery elements 110 may be arranged at approximately 120 degree angles.

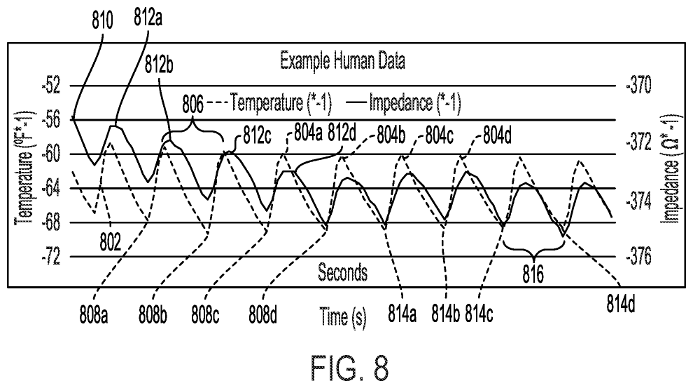

Before, during or after delivery of the energy, the therapeutic assembly 100 may measure, detect, obtain or determine one or more parameters at the treatment site within the blood vessel (606). The one or more parameters may be the temperature of the one or more energy delivery elements 110, the temperature at or near the treatment site and/or the impedance at or near the treatment site. The temperature and/or the impedance may be reflected in a temperature signal or impedance signal that is obtained or determined over a period of time by the therapeutic assembly 100 using the one or more sensors 112 of the renal denervation device 102, as shown in FIG. 8 for example.

The therapeutic assembly 100 may use one or more sensors 112 to measure the temperature of the one more energy delivery elements 110, the temperature at the treatment site along the wall of and/or within the blood vessel and/or measure the impedance at the treatment site within the blood vessel. For example, the therapeutic assembly 100 may measure the voltage change across a voltage gap between two wires within an energy delivery element to determine the temperature. In another example, the therapeutic assembly 100 may measure the impedance of a signal transmitted between two energy delivery elements or sensors, such as the impedance of the signal between an energy delivery element and a sensor, to determine the impedance or use the voltage across the voltage gap and current to calculate the impedance.

The one or more sensors 112 may include multiple temperature sensors or multiple impedance sensors each positioned within a corresponding energy delivery element 110. Each of the multiple temperature sensors or the multiple impedance sensors may independently measure the temperature or the impedance, respectively, at or among the corresponding locations along the wall of the blood vessel 504 that is in contact with the temperature sensor or the impedance sensor, respectively, and/or otherwise within the blood vessel 504. For example, the energy delivery element 110a may be coupled with a sensor 112a at a first location 302a along the blood vessel 304 and the energy delivery element 110b may be coupled with another sensor 112b at a second location 302b along the blood vessel 304. The sensor 112a may measure a first temperature or the first impedance at the first location and the other sensor 112b may measure a second temperature or the second impedance at the second location 502b. Any number of sensors 112 may be used to compute the temperature or the impedance at any number of locations.

The therapeutic assembly 100 may determine the one or more parameters at the treatment site over a period of time. The therapeutic assembly 100 may sample the one or more parameters at a frequency greater than approximately 0.2 to 0.75 Hz, which is the frequency band of interest for vasomotion. This ensures that the sampling captures enough samples of the parameter to provide enough fidelity to capture and monitor changes in the one or more parameters over the period of time.

The therapeutic assembly may calculate or determine one or more parameter values related to the one or more parameters (608). The one or more parameter values are related to, correspond to and/or are based on the one or more parameters. The one or more parameter values may be calculated from the one or more parameters. For example, the one or more parameter values may be a peak (or maximum inflection point), a valley (or minimum inflection point), and/or a derivative (or rate of change) of the corresponding parameter, such as the temperature or impedance at the treatment site over the period of time.

The therapeutic assembly 100 may use the temperature signal and/or the impedance signal to calculate other parameter values that correspond with the temperature signal and/or the impedance signal. The temperature may be determined at multiple locations along the wall of the blood vessel by multiple sensors or may be determined at a single location along the wall of the blood vessel by a single sensor. The therapeutic assembly 100 may calculate or determine when the temperature 802 reaches one or more peak temperatures 804a-d based on the temperature 802 over a period of time. The one or more peak temperatures 804a-d may be a maximum inflection point where the temperature 802 reaches a maximum and changes from an increasing temperature to a decreasing temperature over the period of time. The therapeutic assembly 100 may calculate or determine when the temperature 802 reaches one or more valley temperatures 808a-d. The one or more valley temperatures 808a-d may be a minimum inflection point where the temperature 802 reaches a minimum and changes from a decreasing temperature to an increasing temperature over the period of time.

The therapeutic assembly 100 may calculate or determine when the impedance 810 reaches one or more peak impedances 812a-d. The one or more peak impedances 812a-d may be a maximum inflection point where the impedance 810 increases to a maximum and subsequently decreases. The therapeutic assembly 100 may calculate or determine when the impedance 810 reaches one or more valley impedances 814a-d. The one or more valley impedances 814a-d may be a minimum inflection point where the impedance 810 decreases to a minimum and subsequently increases.

The therapeutic assembly 100 may calculate a first or a second derivative of the temperature that indicates the rate of change of the temperature and/or a first or a second derivative of the impedance that indicates the rate of change of the impedance. For example, the therapeutic assembly 100 may calculate a first derivative of the temperature 802. The first derivative may be the slope between two or more temperatures measured at different times, which may indicate the rate of change of the temperature 802. In another example, the therapeutic assembly 100 may calculate a first derivative of the impedance 810. The first derivative may be the slope between two or more impedances measured at different times, which may indicate the rate of change of the impedance 810.

Once the therapeutic assembly calculates or determines when the one or more parameters reaches two or more peaks and/or two or more valleys, the therapeutic assembly 100 may calculate or determine the frequency of the one or more parameters (610). The therapeutic assembly 100 may calculate or determine the frequency based on the one or more parameters and/or the one or more parameter values to more accurately determine the frequency of the one or more parameters. The frequency of the one or more parameters may refer to the actual frequency or other frequency domain parameter, such as a Fourier Transform, FFT power spectral density, amplitude, phase and/or coherence.

The therapeutic assembly 100 may measure the time period between two or more sequential peak temperatures 804a-d and/or peak impedances 812a-d. The calculated or determined time period may be associated with and correspond to the temperature change cycle 806 and/or the impedance change cycle 816. A sequential peak temperature or impedance is a subsequent peak temperature or impedance that immediately follows a current peak temperature or impedance, respectively, and a sequential valley temperature or impedance is a subsequent valley temperature or impedance that immediately follows a current valley temperature or impedance, respectively. The peaks and valleys of the temperature and/or impedance reflect the contraction and relaxation of the heart muscle, such as between systole and diastole. By measuring the cycle time of the temperature and/or impedance, the therapeutic assembly 100 may calculate the frequency of the temperature and/or impedance, which may be the inverse of the cycle time of the temperature and/or impedance, respectively. The oscillations of the temperature and/or impedance signals collected from the one or more sensors 112 may indicate a cardiac synchronous phasic oscillation. The oscillations may originate from distinct sources, such as the flow of the blood and the arterial pressure of the blood.

The therapeutic assembly 100 may correlate the one or more parameters to the flow of blood within the vessel 304 (612). The therapeutic assembly 100 may correlate the temperature, such as the frequency of the temperature and/or the temperature at the energy delivery element 110 and/or at or near the treatment site, to the flow of blood over the period of time. As mentioned above, the oscillation of the temperature signal over a period of time during ablation may be likely due to the phasic flow of the blood, which may cool the one or more energy delivery elements 110 during high flow systole as opposed to lower flow diastole. When the heart is pumping, during systole, which is the phase of the heartbeat when the heart muscle contracts and pumps blood from the chambers into the arteries, there is a pulse of blood through the blood vessel that enhances the heat transfer and cools the one or more energy delivery elements 110 and/or the one or more sensors 112, which defines a minimum temperature of the temperature cycle 806. During diastole, which is the phase of the heartbeat when the heart muscle relaxes and allows the chambers to fill with blood, blood flow is at a minimum and convective cooling is at its least effective, which defines a maximum temperature of the temperature cycle 806. Thus, the temperature at the treatment site fluctuates and is inversely proportional to the oscillation of the phasic flow of the blood.

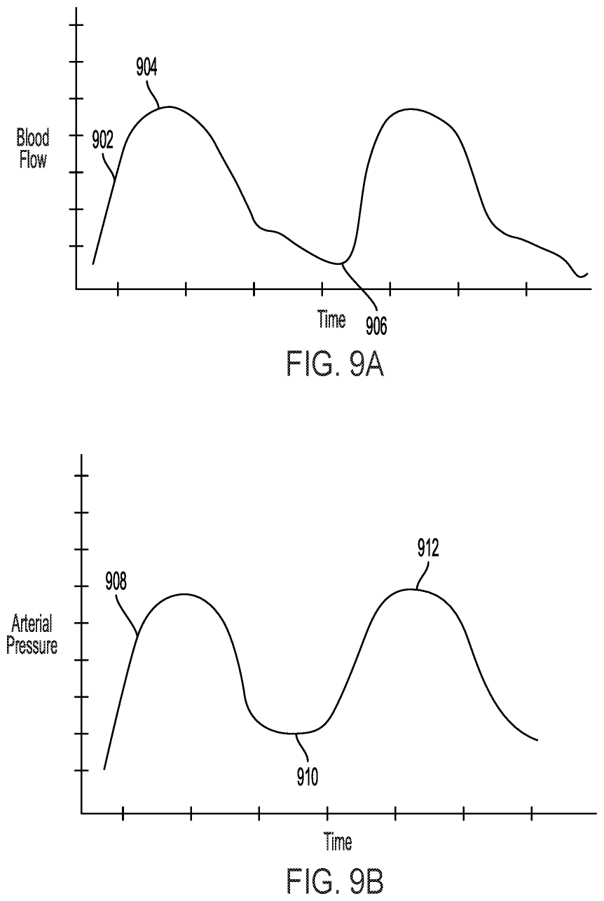

As shown in FIG. 9A, for example, even though the blood flow 902 is inversely proportional to the temperature, the blood flow 902 may have the same frequency of oscillation as the temperature 802 because when the temperature 802 reaches a peak 804a-d, the blood flow 902 will be at a valley 906, and when the temperature 802 reaches a valley 808a-d, the blood flow 902 will be at a peak 904. Thus, there is an inverse relationship between the blood flow and the temperature, and moreover, the frequencies may be similar.

The therapeutic assembly 100 may correlate one or more parameters to the arterial pressure of the blood (614). The arterial pressure of the blood results from the pressure exerted by the blood in the arteries or other blood vessel. The therapeutic assembly 100 may correlate the one or more parameters, such as the impedance and/or the frequency of the impedance between energy delivery elements 110 and/or between the energy delivery element and a sensor at or near the treatment site, to the arterial pressure of the blood over the period of time. The impedance may change during the cardiac cycle within a blood vessel 304 due to compliance-mediated changes in arterial diameter during the cardiac cycle. The oscillation of the impedance signal over the period of time may relate to the phasic flow of the blood, which causes the arterial diameter of the blood vessel 304 to change. As the heart contracts, during systole, the heart pushes blood into the blood vessel 304, and there is a relative increase in the blood volume in the region of the energy delivery element 110 due to the increased arterial diameter during systole, which results in a decreased impedance. Whereas, as the heart relaxes, the heart draws blood back into the heart, and there is a relative decrease in the blood volume in the region of the energy delivery element 110 due to the decreased arterial diameter, which results in an increased impedance. Thus, the impedance is inversely proportional to the change in the vessel diameter, which corresponds to renal artery pressure.

Moreover, as blood flows into the blood vessel 304 and expands the blood vessel 304, the ratio of the surface area of the one or more energy delivery elements 110 that is contact with the blood will be greater because the one or more energy delivery elements 110 that are positioned against the wall of the blood vessel 304 are no longer pressed against the wall of the blood vessel 304 as much in comparison to when the blood vessel 304 is constricted. As a result, the impedance decreases. When the blood vessel 304 constricts because blood is withdrawn into the heart, the ratio of the surface area of the one or more energy delivery elements 110 that is contact with the blood will be less because the one or more energy delivery elements 110 will be pressed against the wall of the blood vessel 304 and not exposed to the blood as much in comparison to when the blood vessel 304 expands. As a result, the impedance increases. This accounts for the tenting of the renal denervation device 102 within the blood vessel 304.

As shown in FIG. 9B, for example, even though the arterial pressure 908 is inversely proportional to the impedance 810, the arterial pressure 908 may have the same frequency of oscillation as the impedance 810 because when the impedance 810 reaches a peak 812a-d, the arterial pressure 908 will be at a valley 910, and when the impedance reaches a valley 814a-d, the arterial pressure 908 will be at a peak 912. Thus, there is an inverse relationship between arterial pressure 908 and impedance, and moreover, the frequencies may be similar.

In some implementations, therapeutic assembly 100 determines or calculates the first derivative of the temperature 802 and/or the first derivative of the impedance 810, the therapeutic assembly 100 may obtain one or more corresponding thresholds and compare the first derivative of the temperature 802 and/or the first derivative of the impedance 810 to the one or more corresponding thresholds. The threshold may be a threshold temperature rate (e.g., degrees Celsius or Fahrenheit/second) and/or the threshold may be a threshold impedance rate (e.g., ohms/second). The therapeutic assembly 100 may determine when the first derivative of the temperature 802 increases past the threshold temperature rate and/or when the first derivative of the impedance 810 increases past the threshold impedance rate to identify the change in the flow of the blood and/or the change in the arterial pressure of the blood, respectively.

By correlating the temperature to the blood flow and/or the impedance to the arterial pressure, the therapeutic assembly 100 does not need to measure the true blood flow or true arterial pressure signal. And so, no calibration of the renal denervation device 102 is necessary because the morphometry of the temperature and/or impedance signals may be proportionate to the blood flow or arterial pressure.

The therapeutic assembly 100 determines the renal sympathetic vasomotion of the blood vessel 304 (616). The therapeutic assembly 100 may determine the renal sympathetic vasomotion, such as the frequency of the oscillation between the contraction and relaxation of the blood vessel 304, based on the flow of blood and/or the arterial pressure of the blood. As the flow of blood and/or the arterial pressure of the blood oscillate, the diameter of the walls of the blood vessel also oscillate, and so, the flow of the blood and/or the arterial pressure of the blood correlate to the renal sympathetic vasomotion of the blood vessel 304.

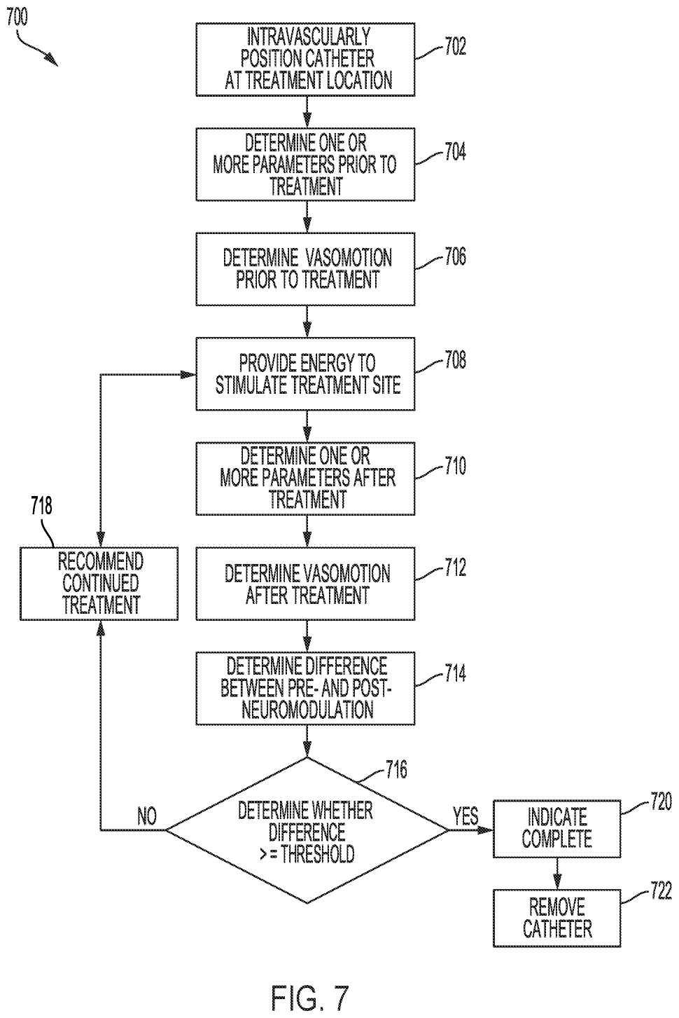

The calculation or determination of the arterial vasomotion may require high signal sampling and/or long data epochs to extract the low frequency information necessary to detect changes in vasomotion. The frequency band of interest for vasomotion is approximately 0.2 to 0.75 Hz. Therefore, measurements may have to be made during steady state periods. This might be achieved by a standby or a low heat mode that would result in electrode temperature fluctuations but not vessel damage. The quantification of the renal sympathetic vasomotion, which measures the rhythmic constriction and relaxation of blood vessels, allows for more accurate treatment of hypertension and may be a biomarker for therapeutic renal denervation, as described further in FIG. 7 for example.

The therapeutic assembly 100 may provide or output an indication that is related to the renal sympathetic vasomotion (618). The therapeutic assembly may provide the indication that is related to the renal sympathetic vasomotion to the clinician. The indication may indicate to the clinician the amount of change in the renal sympathetic vasomotion and/or may characterize the change in the renal sympathetic vasomotion into a recommendation to discontinue or terminate treatment, such as the continued ablation or delivery of neuromodulation energy. The therapeutic assembly 100 may display the indication on the user interface 118 so that the clinician may decide to continue or discontinue treatment. In some implementations, the therapeutic assembly 100 may provide the indication to the renal neuromodulation device 102 and automatically discontinue and/or terminate renal denervation treatment.

FIG. 7 is a flow diagram of a process 700 for determining whether renal neuromodulation is complete and/or was successful. One or more computers or one or more data processing apparatuses, for example, the processor 502 of generator 104 of the therapeutic assembly 100 of FIG. 1, appropriately programmed and/or using one or more other components, such as the one or more sensors 112 and/or the one or more energy delivery elements 110, may implement the process 700.

The renal denervation device 102 including the catheter 108 may be intravascularly delivered and/or positioned within a renal artery of the human patient (702). The catheter 108 may be delivered and/or positioned proximate to nerves innervating a kidney of the human patient. A wire 206 that extends out and away from the distal tip 202 of the catheter 108 may be advanced through the blood vessels to the target location. The wire 206 is then retracted or withdrawn within the distal tip 202, which causes the shape of the catheter 108 to change from the low-profile configuration to the expanded deployed configuration. In the expanded deployed configuration, the one or more energy delivery element 110 may be in apposition to the wall of the blood vessel and be arranged in a generally helical or spiral configuration.

The therapeutic assembly 100 determines, detects or otherwise obtains one or more parameters prior to treatment (704). The therapeutic assembly 100 determines, detects or otherwise obtains the one or more parameters using the one or more sensors 112, as described above. These one or more parameters are pre-neuromodulation parameters that provide a baseline to establish the renal sympathetic vasomotion of the vessel 304 prior to treatment. The therapeutic assembly 100 may be in a standby or low power mode that delivers a small amount of energy so that the one or more parameters may be measured, detected or otherwise obtained.

The one or more parameters may include the temperature at one or more energy delivery elements 110 and/or at or near a treatment site and/or the impedance between two or more energy delivery elements 110 and/or between an energy delivery element 110 or an energy delivery element 110 and a sensor, such as a ground patch. As described above, the one or more parameters may be related to the renal sympathetic vasomotion of the vessel 304. For example, the temperature is inversely proportional to the flow of blood through the blood vessel 304 during diastole and systole, and similarly, the impedance is inversely proportional to the arterial pressure of the blood within the blood vessel 304, which corresponds to the diameter of the blood vessel 304, during systole and systole.

The measurement may be an average of measurements taken over a period of time or over several measurements. The measurements may be taken by one or more of the energy delivery elements 110 over the period of time, such as over a period of seconds (e.g., about 0.5 seconds, about 1 second, about 2 seconds etc.) to account for changes in the temperature. In some implementations, the obtained measurements may be communicated to and stored in the memory 504.

The therapeutic assembly 100 determines the renal sympathetic vasomotion of the blood vessel 304 prior to treatment (706). The therapeutic assembly 100 determines the renal sympathetic vasomotion of the blood vessel 304 prior to treatment (or “pre-neuromodulation renal sympathetic vasomotion) based on the one or more pre-neuromodulation parameters measured, detected or obtained prior to treatment, as described above. This establishes the baseline measurements of the renal sympathetic vasomotion of the blood vessel 304 to which the post-neuromodulation renal sympathetic vasomotion of the blood vessel 304 may be compared to determine efficacy of the renal neuromodulation treatment.

Once the pre-neuromodulation renal sympathetic vasomotion is determined, The therapeutic assembly 100 provides energy to stimulate the treatment site (708). The generator 104 may provide the neuromodulation energy through the one or more energy delivery elements 110 to stimulate the nerves proximate to the treatment site (e.g., the wall of the blood vessel 304). For example, the generator 104 may provide radio frequency (RF) energy to ablate the nerves proximate to the treatment site via one or more energy delivery elements 110, such as one or more electrodes. In some implementations, the therapeutic assembly 100 may provide pulsed electrical energy, microwave energy, optical energy, ultrasound energy (e.g., intravascularly delivered ultrasound and/or high-frequency ultrasound), direct heat energy, radiation, cryogenic cooling, chemical-based treatment, and/or another suitable type of neuromodulation energy.

The therapeutic assembly 100 determines, detects or otherwise obtains one or more parameters after treatment (710). These one or more parameters are post-neuromodulation parameters that used to establish the renal sympathetic vasomotion of the vessel 304 after treatment. The therapeutic assembly 100 determines, detects or otherwise obtains the one or more parameters using the one or more sensors 112, as described above, and may be determined, detected or otherwise obtained over a period of time after treatment, such as immediately after treatment, after approximately a few minutes, a few hours, a few days or other length of time after treatment.

In some implementations, the therapeutic assembly 100 may determine, detect or otherwise obtain the one or more parameters during treatment. For example, if impedance is to be measured during the ablation period, it might be necessary to oscillate the ablation power delivery with the impedance measurement at a very high frequency to avoid ablation electric fields from interfering with impedance measurement electric fields. The sampling of the one or more parameters may occur when energy has been delivered but the therapeutic assembly 100 enters into a standby or a low power or low heat mode. The therapeutic assembly 100 may cycle from energy delivery to the standby or the lower power or low heat mode during treatment so that measurements of the one or more parameters may be sampled.

The one or more post-neuromodulation parameters may be obtained in generally the same manner as the pre-neuromodulation parameters. Similarly, the one or more post-neuromodulation parameters may be measured as a single measurement or as multiple measurements over a period of time. The post-neuromodulation parameters may be communicated to and stored in the memory 504.

The therapeutic assembly 100 determines the renal sympathetic vasomotion of the vessel 304 after treatment (712). The therapeutic assembly 100 determines the renal sympathetic vasomotion, such as changes in the sympathetically mediated arteriolar vasomotion, of the vessel 304 immediately after treatment (hereinafter, referred to as “post-neuromodulation renal sympathetic vasomotion) based on the one or more post-neuromodulation parameters measured, detected or obtained after or during treatment. The determination of the renal sympathetic vasomotion is based on the inverse relationship of the one or more parameters to the blood flow and/or arterial pressure, as described above. This establishes the measurements of the renal sympathetic vasomotion of the blood vessel 304 after treatment to determine the efficacy of the treatment and/or the success or failure of the treatment after the neuromodulation energy is delivered to the treatment site.

Once the post-neuromodulation renal sympathetic vasomotion of the blood vessel 304 is determined, the therapeutic assembly 100 determines the difference between the post-neuromodulation renal sympathetic vasomotion and the pre-neuromodulation renal sympathetic vasomotion (714). The therapeutic assembly 100 compares the pre- and post-neuromodulation measurements of the renal sympathetic vasomotion and determines the amount of change or delta in the amount of oscillation of the vessel wall pre- and post-neuromodulation to determine whether the renal denervation treatment was or is successful and whether further neuromodulation treatment should be employed or recommended to the clinician.

The determined difference may, for example, may be computed or calculated automatically and the determined difference may be measured as an absolute measurement or as a relative percentage. The determined difference may be reflective of a change in the renal sympathetic vasomotion, which may correspond to a reduction in blood pressure or other parameter that relates to hypertension, and may be used to assess the efficacy of the neuromodulation treatment.

The therapeutic assembly 100 determines whether the difference between the pre- and post-renal sympathetic renal sympathetic vasomotion is greater than or equal to a threshold (716). The therapeutic assembly 100 may obtain the threshold from the memory 504, which may have been stored via user-input and/or from a historical analysis of a population sized data set of renal sympathetic vasomotion parameters and their corresponding successful or failed renal neuromodulation procedures. The threshold may be, for example, equivalent to a percentage change in renal sympathetic vasomotion, such as approximately 5% reduction, 10% reduction, and/or 30% reduction, etc. The different thresholds may represent different levels of success, partial success or failure.

The therapeutic assembly 100 compares the difference to the threshold. If the difference is greater than or equal to the threshold, the therapeutic assembly 100 may determine that the neuromodulation treatment is complete and/or was successful. Otherwise, if the difference is less than or equal to the threshold, the therapeutic assembly 100 may determine that the neuromodulation treatment is not complete and/or was not successful.

When the therapeutic assembly 100 determines that the neuromodulation treatment is not complete and/or was not successful, the therapeutic assembly 100 may indicate to the clinician that the neuromodulation treatment is not complete and/or that the neuromodulation treatment was not successful (718). The indication may include a recommendation to continue neuromodulation treatment and/or may automatically continue neuromodulation treatment. The therapeutic assembly 100 may require confirmation from the clinician prior to continuing neuromodulation treatment. For example, the clinician may reposition the renal denervation device prior to the subsequent ablation. Once confirmed and/or when done automatically, the therapeutic assembly 100 may store the post-neuromodulation renal sympathetic vasomotion parameters as the baseline or pre-neuromodulation renal sympathetic vasomotion parameters and provide neuromodulation energy to stimulate the treatment site or another treatment site.

When the therapeutic assembly 100 determines that the neuromodulation treatment is complete and/or was successful, the therapeutic assembly 100 may indicate to the clinician that the neuromodulation treatment is complete and that the neuromodulation treatment was or is successful (720). The therapeutic assembly 100 may provide or display an indication on the user interface 118 that the neuromodulation treatment is complete or was successful, which allows the clinician to decide to continue neuromodulation treatment or to terminate neuromodulation treatment. In some implementations, the therapeutic assembly 100 may provide a different indicator, such as an audio or other visual indicator, that the neuromodulation treatment is complete or was successful. The visual indicator may be a light, such as a red light-green light, that indicates whether RDN is complete.

The indication may also inform the clinician to remove the catheter 108. If the clinician elects to continue another round of neuromodulation treatment, the post-neuromodulation renal sympathetic vasomotion may be stored as the baseline for the next round of treatment. The clinician may reposition the renal denervation device 102 prior to the next round of treatment. And, the clinician may proceed with an additional round of neuromodulation treatment. Otherwise, if the clinician decides that that neuromodulation treatment is complete and/or successful, the clinician may subsequently remove the catheter 108 (722). By providing real-time feedback of the completion and/or success or failure of the neuromodulation treatment, the therapeutic assembly 100 performs the evaluation of the neuromodulation treatment in real-time without requiring additional equipment or devices and without the need to insert, move or otherwise reposition the catheter 108, which reduces the complexity of the neuromodulation procedure for the clinician.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A therapeutic assembly for renal denervation, comprising:
a sensor configured to detect a temperature or an impedance at a location within a vessel over a period of time; and
a processor coupled to the sensor and configured to:
correlate the temperature or the impedance to a flow of blood within the vessel or an arterial pressure of the blood within the vessel, and
determine vasomotion of a wall of a distal vessel based on the flow of blood or the arterial pressure of the blood.

2. The therapeutic assembly of claim 1, wherein the processor is configured to:
determine a frequency of the temperature or the impedance over the period of time; and
correlate the temperature or the impedance to the flow of blood within the vessel or the arterial pressure of the blood using the frequency of the temperature or the impedance.

3. The therapeutic assembly of claim 1, further comprising:
an energy delivery element configured to deliver neuromodulation energy to a wall of the vessel.

4. The therapeutic assembly of claim 3, wherein the processor is configured to:
determine the vasomotion of the wall of the distal vessel based on the flow of blood or the arterial pressure of the blood prior to the delivery of the neuromodulation energy; and
determine the vasomotion of the wall of the distal vessel after the delivery of the neuromodulation energy.

5. The therapeutic assembly of claim 4, wherein the processor is configured to:
compare the vasomotion of the wall prior to the delivery of the neuromodulation energy and the vasomotion of the wall after the delivery of the neuromodulation energy; and
determine a success or failure of renal neuromodulation based on the comparison.

6. The therapeutic assembly of claim 1, wherein to correlate the temperature or the impedance to the flow of blood or the arterial pressure of the blood the processor is configured to correlate the temperature to the flow of blood within the vessel.

7. The therapeutic assembly of claim 6, wherein to correlate the temperature to the flow of blood within the vessel the processor is configured to:
measure or detect, using the sensor, variations in the temperature at a location on a wall within the vessel over the period of time; and
associate the variations in the temperature with the flow of blood, wherein the variations in the temperature are inversely proportional to the flow of the blood.

8. The therapeutic assembly of claim 1, wherein to correlate the temperature or the impedance to the flow of blood or the arterial pressure of the blood the processor is configured to correlate the impedance to the arterial pressure of the blood within the vessel.

9. The therapeutic assembly of claim 8, wherein to correlate the impedance to the arterial pressure of the blood within the vessel the processor is configured to:
measure or detect, using the sensor, variations in the impedance at a location on a wall within the vessel over the period of time; and
associate the variations in the impedance with the arterial pressure of the blood within the vessel, wherein the variations in the impedance are inversely proportional to a diameter of the vessel that corresponds to the arterial pressure of the blood.

10. A therapeutic assembly for renal denervation, comprising:
a first sensor configured to detect a temperature at a location on a wall of a vessel over a period of time;
a second sensor configured to detect an impedance at the location on the wall of the vessel over the period of time; and
a processor coupled to the first sensor and the second sensor and configured to:
correlate the temperature to a flow of blood within the vessel,
correlate the impedance to an arterial pressure of the blood within the vessel, and
determine vasomotion of the wall of the vessel based on the flow of blood and the arterial pressure of the blood.

11. The therapeutic assembly of claim 10, wherein the processor is configured to:
determine a frequency of the temperature and a frequency of the impedance at the location of the wall of the vessel over the period of time;
correlate the temperature to the flow of blood based on the frequency of the temperature; and
correlate the impedance to the arterial pressure based on the frequency of the impedance.

12. The therapeutic assembly of claim 10, further comprising:
an energy delivery element configured to deliver neuromodulation energy to the location of the wall of the vessel.

13. The therapeutic assembly of claim 12, wherein the processor is configured to:
determine the vasomotion of the wall of the vessel based on the flow of blood and the arterial pressure of the blood prior to the delivery of the neuromodulation energy; and
determination the vasomotion of the wall of the vessel after the delivery of the neuromodulation energy.

14. The therapeutic assembly of claim 13, wherein the processor is configured to:
compare the vasomotion of the wall prior to the delivery of the neuromodulation energy and the vasomotion of the wall after the delivery of the neuromodulation energy;
determine a success or failure of renal neuromodulation based on the comparison; and
provide an indication of the success or failure of the renal neuromodulation.

15. The therapeutic assembly of claim 14, further comprising:
a display configured to output the indication of the success or failure of the renal neuromodulation to an operator;
wherein the processor is configured to:
cause the display to output the indication of the success or failure of the renal neuromodulation to the operator.

16. The therapeutic assembly of claim 14, wherein to determine the success or failure of the renal neuromodulation based on the comparison the processor is configured to:
determine a difference between the vasomotion of the wall prior to the delivery of the neuromodulation energy and the vasomotion of the wall after the delivery of the neuromodulation energy; and
determine whether the difference is greater than or equal to a threshold amount.

17. The therapeutic assembly of claim 10, wherein variations in the temperature over the period of time are inversely proportional to the flow of the blood and variations in the impedance over the period of time are inversely proportional to a diameter of the vessel that corresponds to the arterial pressure of the blood.

18. A method of renal denervation, comprising:
detecting, by a first sensor, a temperature at a location of a wall of a vessel over a period of time;
detecting, by a second sensor, an impedance at the location of the wall of the vessel over the period of time;
correlating, by a processor, the temperature to a flow of blood within the vessel;
correlating, by the processor, the impedance to an arterial pressure of the blood within the vessel; and
determining, by the processor, vasomotion of the wall of the vessel based on the flow of blood and the arterial pressure of the blood.

19. The method of claim 18, comprising:
determining, by the processor, a frequency of the temperature and a frequency of the impedance at the location of the wall of the vessel over the period of time;
correlating, by the processor, the temperature to the flow of blood within the vessel based on the frequency of the temperature; and
correlating, by the processor, the impedance to the arterial pressure of the blood within the vessel based on the frequency of the impedance.

20. The method of claim 19, further comprising:
providing, by the processor, an indication of a success or failure of the renal neuromodulation based on the vasomotion of the wall of the vessel.
